(19) United States
(12) Patent Application Publication
(10) Pub. No.: US 2026/0248678 A1
(43) Pub. Date: Aug. 27, 2026

(54) SCRAPPING MASSAGE DEVICE WITH DIAGNOSTIC FUNCTION AND SCRAPPING MASSAGE METHOD USING SAME

(71) Applicant: APR CO., LTD., Seoul (KR)

(72) Inventors: Jong Chul Ji, Seoul (KR); Gyoun Jung Lee, Seoul (KR); Seung Woo Park, Seoul (KR)

(21) Appl. No.: 19/160,336

(22) PCT Filed: Jul. 2, 2025
(86) PCT No.: PCT/KR24/01841

§ 371 Date: Aug. 27, 2025

(30) Foreign Application Priority Data
Mar. 2, 2023 (KR) 10-2023-0027736
Mar. 2, 2023 (KR) 10-2024-0013064

(51) Int. Cl. A61H 7/00; A61H 39/00; A61H 39/04

(52) U.S. Cl. CPC A61H 7/003; A61H 7/005; A61H 7/007; A61H 39/002; A61H 39/04; A61H 2201/0153; A61H 2201/0165; A61H 2201/0188; A61H 2201/5043; A61H 2201/5048; A61H 2201/5071

(57) ABSTRACT

The present invention relates to a scrapping massage device and a scrapping massage method and, more specifically, to a scrapping massage device with a diagnostic function that allows for safer scrapping massage treatment while protecting the skin of a subject, and a scrapping massage method using same. The scrapping massage device with a diagnostic function, according to one aspect of the present invention, comprises a diagnostic module that constitutes a main body and checks the level of pain in a portion to be treated. The scrapping massage method using the scrapping massage device with a diagnostic function, according to one aspect of the present invention, comprises: a pain diagnosis step; a pain level checking step; and a scrapping massage treatment step.

### TECHNICAL FIELD

The present disclosure relates to a Gua sha massage apparatus and a Gua sha massage method, and more particularly, to a Gua sha massage apparatus with a diagnostic function capable of performing a safer Gua sha massage treatment while protecting the skin of a subject, and a Gua sha massage method using the same.

### BACKGROUND ART

Generally, massage refers to rubbing, tapping, or kneading the skin with hands to improve skin elasticity or relieve muscle fatigue, and may be performed using bare hands or instruments.

Gua sha massage (Gua sha therapy), which is one type of massage, is a traditional folk physiotherapy handed down from China, in which various tools are used to repeatedly press or rub along meridians and acupoints to loosen the clumped area and promote blood circulation.

A Gua sha massage apparatus for performing such Gua sha massage generally includes a handle configured to be gripped by the operator, and a protrusion provided on one side the handle in various shapes, the protrusion being configured to allow repeated pressing or rubbing of various points of the body.

For example, Korean Utility Model Publication No. 20-2022-0002575 discloses a Gua sha massage apparatus including: a housing having an upper surface and a lower surface, and four side surfaces between the upper surface and the lower surface; a low-frequency generator provided on a first side surface which is one side surface among the four side surfaces and configured to generate a low frequency wave; and a high-frequency generator provided on a second side surface which is one side surface among the four side surfaces and configured to generate a high frequency wave.

In addition, the apparatus has protrusions of various shapes to perform Gua sha massage by utilizing a stimulus generated by scraping the skin with strong pressure through the protrusions.

On the other hand, since Gua sha massage apparatuses are known to be relatively safe, people tend to apply stronger pressure while scraping the skin using the Gua sha massage apparatus to obtain stronger stimulation.

However, it has not been scientifically proven that strong friction with the skin using strong pressure improves the skin condition; but rather, it may cause skin damage or induce excessive tension of the surrounding muscles, leading to pain.

In addition, depending on the condition of a person's skin, the magnitude of force that can be tolerated varies, and some people have skin that may tear easily even with slight stimulation.

Such individuals are cases in which tissue supporting the skin and blood vessels is little or absent, which makes them prone to bruising when pressure is applied.

Especially online, bruising (ecchymosis) caused by strong pressure is incorrectly described as a good “stagnant blood” phenomenon resulting from toxin release, which, as described above, is a scientifically unfounded expression and is merely a phenomenon in which blood pools due to ruptured blood vessels.

In spite of these problems, users who use Gua sha massage apparatuses often fail to maintain an appropriate pressure suitable for their skin and, in an unconscious attempt to feel the refreshing sensation from stimulation, gradually apply increasingly strong pressure during the Gua sha massage.

Consequently, strong friction caused by strong pressure inevitably results in bruising or skin tears on the subject's skin, as well as pain due to excessive tension of surrounding muscles.

Accordingly, there is an urgent need to develop a Gua sha massage apparatus and method capable of performing a safer Gua sha massage treatment while protecting the skin of the subject receiving the treatment.

### DISCLOSURE

### Technical Problem

The present disclosure has been made to solve the above problems, and an object thereof is to provide a Gua sha massage apparatus with a diagnostic function capable of performing a safer Gua sha massage treatment while protecting the skin of a subject receiving the treatment, and a Gua sha massage method using the same.

It is another object of the present disclosure to provide a Gua sha massage apparatus with a diagnostic function and a Gua sha massage method using the same, which, by generating and notifying feedback recognizable by an operator or a subject according to pain pressure values measured by the pressure measurer of the diagnosis module, can significantly reduce the degree and likelihood of bruising (ecchymosis) that may occur at the portion to be treated during the Gua sha massage, thereby protecting the subject's skin and enabling a safe massage treatment.

In other words, an object of the present disclosure is to provide a Gua sha massage apparatus with a diagnostic function and a Gua sha massage method using the same, which can perform a traditional Gua sha massage function using friction stimulation, and which also performs a diagnostic function of measuring pain pressure values via a diagnosis module so as to protect the skin of a subject already under strong scratching pressure and to enable a safer Gua sha massage treatment.

The problems of the present disclosure are not limited to those mentioned above, and other problems not mentioned will be clearly understood by those of ordinary skill in the art from the following description.

### Technical Solution

According to an aspect of the present disclosure, a Gua sha massage device with a diagnostic function is provided.

The Gua sha massage apparatus with the diagnostic function includes a main body including a graspable handle and at least one skin contact having a contact surface configured to be frictionally in contact with a portion to be treated.

In this case, the main body includes a diagnosis module configured to measure a pressure value generated upon contact with the portion to be treated and to confirm a level of pain at the portion to be treated by using the pressure value.

In this case, the diagnosis module includes at least one pressure measurer configured to measure the pressure value generated upon contact with the portion to be treated; a database configured to store information on a pain pressure value confirmed by using the pressure value measured by the pressure measurer and the pressure value measured by the pressure measurer; a comparator configured to determine the pain pressure value and compare the pain pressure value with the pressure value measured by the pressure measurer to determine a numerical value; a controller configured to generate a control signal based on the determination of the comparator; and a feedback generator configured to generate a notification signal recognizable by an operator based on the control signal from the controller.

Meanwhile, in an embodiment, the pressure measurer includes: a diagnostic member formed on one side of the skin contact, the diagnostic member having a diagnostic surface configured to come into contact with the portion to be treated and being integrally provided with the skin contact; a pressure sensor disposed in an internal space of the skin contact and configured to measure the pressure value transmitted through the diagnostic member; and an elastic member disposed in the internal space of the skin contact and configured to accommodate a displacement according to a pressure applied to the diagnostic member and to support the pressure sensor.

Meanwhile, in an embodiment, the pressure sensor includes a load cell.

Meanwhile, the diagnostic member may include a first diagnostic member having the diagnostic surface; and a second diagnostic member disposed inside the first diagnostic member and configured to support the load cell and the elastic member, wherein the first diagnostic member and the second diagnostic member have different hardnesses.

Preferably, the pressure measurer is disposed along a line in a direction the same as a pressure direction applied to a portion to be treated of a subject.

Meanwhile, the main body may have a donut shape or a knuckle shape with a through hole through which a finger of a subject passes.

Meanwhile, the pain pressure value may be an average pressure value of pressure values measured for each unit of time while pressing the portion to be treated for a preset period of time, or a maximum pressure value among pressure values measured while pressing the portion to be treated for a preset period of time.

Meanwhile, the main body further includes an operation button on one side thereof. In this case, the pain pressure value may be a pressure value measured by the pressure measurer when the operation button is operated.

Meanwhile, the main body includes at least one of a vibration motor, a speaker, a light-emitting lamp, and a numerical display on one side thereof.

And, the information stored in the database is linked to an external smartphone or server via a network.

Meanwhile, the skin contact includes an electrical muscle stimulation (EMS) stimulation module configured to stimulate a muscle with a microcurrent or a medium-frequency current of a preset frequency, or a radio frequency (RF) stimulation module configured to provide deep thermal stimulation using a frequency.

According to another aspect of the present disclosure, a Gua sha massage method using the Gua sha massage apparatus with the diagnostic function is provided.

The Gua sha massage method using the Gua sha massage apparatus with the diagnostic function includes a pain diagnosis step of bringing the pressure measurer into contact with the portion to be treated, pressing the portion to be treated, and storing the pressure value measured by the pressure measurer as the pain pressure value when pain occurs at the portion to be treated; a pain score confirmation step of converting the pain pressure value into a pain scale, scoring it, and storing the score; and a Gua sha massage treatment step of performing a Gua sha massage within a range of pressure values equal to or less than the pain pressure value.

In this case, the pain diagnosis step includes a pressing step of bringing the pressure measurer into contact with the portion to be treated of a subject and pressing the portion to be treated using the pressure measurer; a press stopping step of stopping the pressing when pain is confirmed at the portion to be treated; a pain confirmation step of confirming the pain level of the subject for the pressure value measured in the press stopping step; and a pain pressure value storing step of storing the pressure value confirmed in the pain confirmation step as the pain pressure value.

In this case, the pain pressure value storing step includes storing, as the pain pressure value, an average pressure value of pressure values measured for each unit of time while pressing the portion to be treated for a preset period of time, or storing, as the pain pressure value, a maximum pressure value among pressure values measured while pressing the portion to be treated for a preset period of time.

In addition, the pain pressure value storing step may include storing, as the pain pressure value, a selected pressure value when an operation button provided on one side of the main body of the Gua sha massage apparatus with the diagnostic function is pressed.

Meanwhile, the pain score confirmation step includes a pain scale conversion step of confirming a pain score by comparing the pain pressure value with a score in a pain scale chart; and a pain score storing step of storing the pain score.

In this case, in one embodiment, the pain scale chart may employ a numerical rating scale (NRS) pain scale.

Meanwhile, the Gua sha massage treatment step includes a pressure massage step of pressing while bringing the skin contact into contact with the portion to be treated; and a pain notification step of generating a notification signal recognizable by the operator or a subject through the feedback generator when the pressure value measured by the pressure measurer is equal to or greater than the stored pain pressure value.

And, the Gua sha massage treatment step further includes a pain relief step of operating any one of an electrical muscle stimulation (EMS) stimulation module configured to stimulate a muscle with a microcurrent or a medium-frequency current of a preset frequency, or a radio frequency (RF) stimulation module configured to provide deep thermal stimulation using a frequency to stimulate the portion to be treated.

Meanwhile, the notification signal is generated as any one of a numerical display signal, a sound signal, a vibration signal, or a light-emitting signal.

And, in the pain notification step, when the pressure value measured by the pressure measurer increases to be equal to or greater than the stored pain pressure value, the numerical display signal may indicate a change in numerical value, the sound signal may indicate a change in volume, the vibration signal may indicate a change in intensity, and the light-emitting signal may indicate a change in blinking frequency of light.

### Advantageous Effects

According to the configuration above, the Gua sha massage apparatus with a diagnostic function and the Gua sha massage method using the same according to the present disclosure can perform Gua sha massage within a range of pain pressure values measured by the pressure measurer of the diagnosis module, thereby protecting the subject's portion to be treated and enabling a safe massage treatment.

In addition, when a pressure value equal to or greater than the pain pressure value is generated, feedback that can be confirmed by the operator or the subject is generated and notified through the feedback generator of the diagnosis module, thereby significantly reducing the degree and likelihood of bruising (ecchymosis) that may occur at the Gua sha massage portion to be treated, protecting the skin of the portion to be treated and enhancing safety.

In addition, the feedback generator notifies the operator or the subject with any one of numerical display, sound, light, or vibration notification signals, which not only prevents excessive use of the Gua sha massage apparatus but also prevents damage to the subject's portion to be treated.

In addition, the pain pressure value can be specified through manual or automatic operation, enabling the operator to perform the Gua sha massage treatment within a desired pressure range.

In addition, the pressure measurer of the diagnosis module can accurately measure pressure by using a load cell.

In addition, the EMS stimulation module and the RF stimulation module can be selectively used under control, thereby treating a portion to be treated with severe pain to alleviate the pain level and improve the painful area.

In addition, pain pressure values suitable for each operator for each portion to be treated can be confirmed, and the pressure values stored in the database can be converted into data, thereby enabling performance of a safer Gua sha massage treatment with the pressure most suitable for the operator based on the accumulated pressure value data.

In addition, since the operator or subject perceives the level and area of pain through visual, auditory, and tactile senses during the Gua sha massage treatment, the concentration on the Gua sha massage treatment is naturally increased, thereby facilitating management of the painful area through the Gua sha massage.

In addition, the pain pressure values stored after the Gua sha massage treatment can be stored as individual data for each subject, which can serve as useful feedback for the user during the next Gua sha massage treatment, thereby enabling effective Gua sha massage treatment and increasing user satisfaction with the Gua sha massage apparatus.

Advantageous effects of the present disclosure are not limited to the above-described effects and should be understood to include all effects that can be inferred from the configuration of the invention described in the detailed description or claims of the present disclosure.

### DESCRIPTION OF DRAWINGS

FIG. 1 is a schematic diagram showing a Gua sha massage apparatus with a diagnostic function according to an embodiment of the present disclosure.

FIG. 2 is a schematic diagram showing a configuration of a diagnosis module applied to the Gua sha massage apparatus with the diagnostic function according to an embodiment of the present disclosure.

FIG. 3 is a schematic diagram showing an example of a pressure measurer of the diagnosis module applied to the Gua sha massage apparatus with the diagnostic function according to an embodiment of the present disclosure.

FIG. 4 is a schematic diagram showing an example of a notification signal form generated by a feedback generator of the diagnosis module applied to the Gua sha massage apparatus with the diagnostic function according to an embodiment of the present disclosure.

FIG. 5 is a block diagram showing a Gua sha massage method using the Gua sha massage apparatus with the diagnostic function according to an embodiment of the present disclosure.

FIG. 6 is a block diagram showing a pain diagnosis step in the Gua sha massage method using the Gua sha massage apparatus with the diagnostic function according to an embodiment of the present disclosure.

FIG. 7 is a block diagram showing a pain score confirmation step in the Gua sha massage method using the Gua sha massage apparatus with the diagnostic function according to an embodiment of the present disclosure.

FIG. 8 is a view showing an example of a pain scale chart applied to the pain score confirmation step in the Gua sha massage method using the Gua sha massage apparatus with the diagnostic function according to an embodiment of the present disclosure.

FIG. 9 is a block diagram showing a Gua sha massage treatment step in the Gua sha massage method using the Gua sha massage apparatus with the diagnostic function.

### BEST EMBODIMENTS

The present disclosure proposes, in the best form, a Gua sha massage device with a diagnostic function.

The Gua sha massage apparatus with the diagnostic function includes a main body including a graspable handle and at least one skin contact having a contact surface configured to be frictionally in contact with a portion to be treated.

The main body is proposed to include a diagnosis module configured to measure a pressure value generated upon contact with the portion to be treated and to confirm a level of pain at the portion to be treated by using the pressure value.

The diagnosis module is proposed to include at least one pressure measurer configured to measure the pressure value generated upon contact with the portion to be treated; a database configured to store information on a pain pressure value confirmed by using the pressure value measured by the pressure measurer and the pressure value measured by the pressure measurer; a comparator configured to determine the pain pressure value and compare the pain pressure value with the pressure value measured by the pressure measurer to determine a numerical value; a controller configured to generate a control signal based on the determination of the comparator; and a feedback generator configured to generate a notification signal recognizable by an operator based on the control signal from the controller.

The present disclosure proposes, in its best form, a Gua sha massage method using the Gua sha massage apparatus with the diagnostic function.

The Gua sha massage method using the Gua sha massage apparatus with the diagnostic function is proposed to include a pain diagnosis step of bringing the pressure measurer into contact with the portion to be treated, pressing the portion to be treated, and storing the pressure value measured by the pressure measurer as the pain pressure value when pain occurs at the portion to be treated; a pain score confirmation step of converting the pain pressure value into a pain scale, scoring it, and storing the score; and a Gua sha massage treatment step of performing a Gua sha massage within a range of pressure values equal to or less than the pain pressure value.

### DETAILED DESCRIPTION OF THE EMBODIMENTS

Hereinafter, exemplary embodiments of the present disclosure will be described in detail so that those of ordinary skill in the art can readily implement the present disclosure with reference to the accompanying drawings. The present disclosure may be embodied in many different forms and is not limited to the embodiments set forth herein. In the drawings, parts unrelated to the description are omitted for clarity in describing the present disclosure, and throughout the specification, the same or similar reference numerals denote the same or similar elements.

The words and terms used in the present specification and claims should not be construed as being limited to their ordinary or dictionary meanings, but should be construed as having meanings and concepts consistent with the technical spirit of the present disclosure, in accordance with the principle that an inventor may define terms and concepts to best describe the present disclosure.

Accordingly, the embodiments described in the present specification and the configurations shown in the drawings correspond to preferred embodiments of the present disclosure, and do not represent all the technical idea of the present disclosure, so the configurations may have various examples of equivalent and modification that can replace them at the time of filing the present disclosure.

It should be understood that the terms “comprise” or “include” or “have” and the like, when used in the present specification, are intended to specify the presence of stated features, numbers, steps, operations, elements, components, or combinations thereof, but do not preclude the possibility of the presence or addition of one or more other features, numbers, steps, operations, elements, components, or combinations thereof.

The presence of an element in/on “front”, “rear”, “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, the expression that an element is “connected” to another element should be construed to include not only a case of being directly connected to each other but also a case of being indirectly connected to each other.

In addition, in describing the present disclosure, detailed descriptions of related known functions or configurations will be omitted so as not to obscure the gist of the present disclosure.

Hereinafter, an embodiment of a Gua sha massage apparatus having a diagnostic function and a Gua sha massage method using the same according to the present disclosure will be described with reference to the drawings.

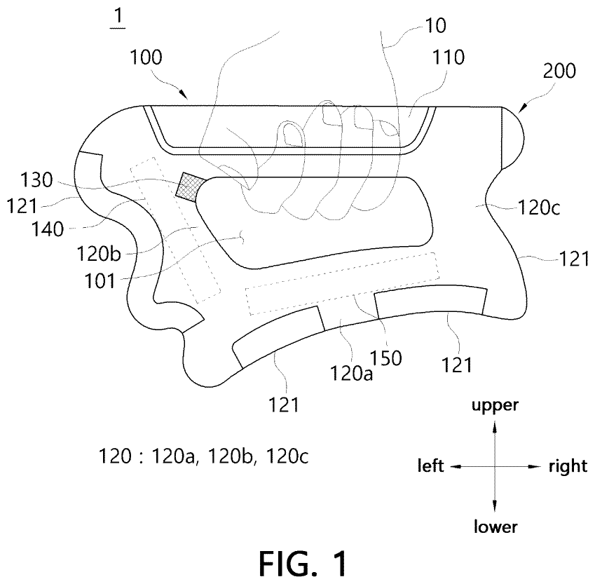

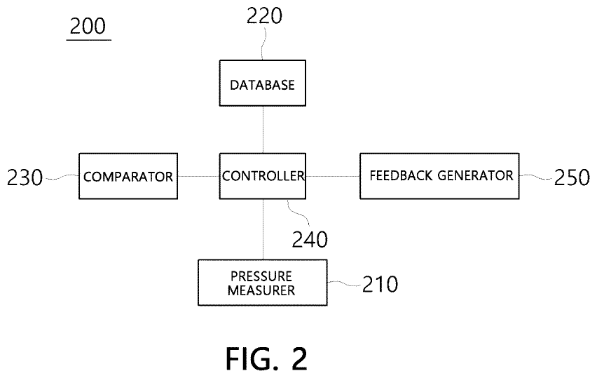

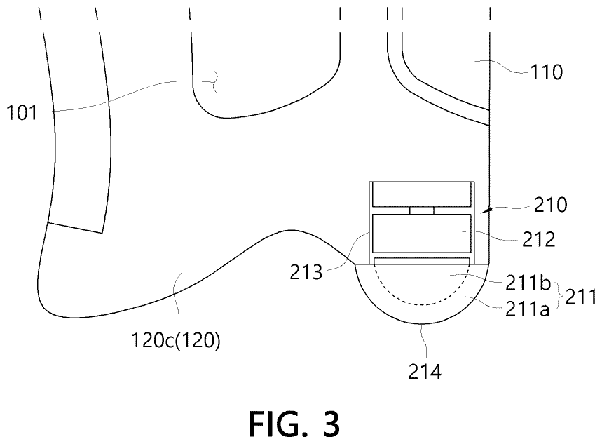

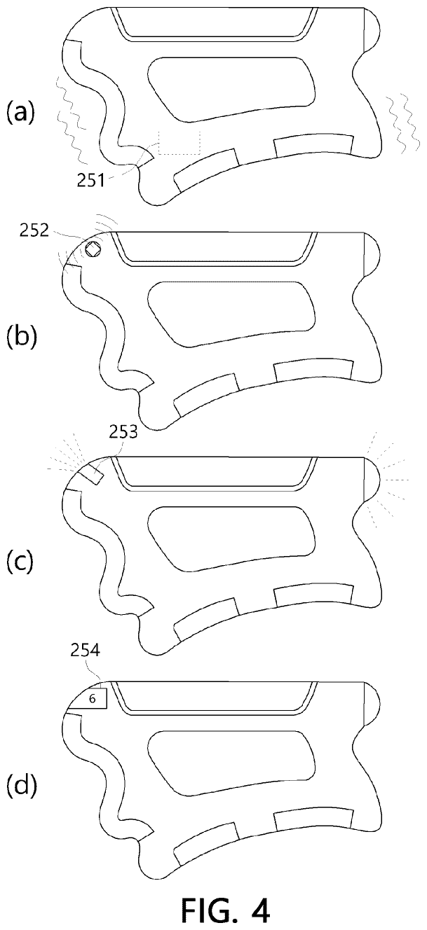

FIG. 1 is a schematic diagram showing a Gua sha massage apparatus with a diagnostic function according to an embodiment of the present disclosure, FIG. 2 is a schematic diagram showing a configuration of a diagnosis module applied to the Gua sha massage apparatus with the diagnostic function according to an embodiment of the present disclosure, FIG. 3 is a schematic diagram showing an example of a pressure measurer of the diagnosis module applied to the Gua sha massage apparatus with the diagnostic function according to an embodiment of the present disclosure, and FIG. 4 is a schematic diagram showing an example of a notification signal form generated by a feedback generator of the diagnosis module applied to the Gua sha massage apparatus with the diagnostic function according to an embodiment of the present disclosure.

As illustrated, the Gua sha massage apparatus 1 with a diagnostic function according to an embodiment of the present disclosure enables an operator, during a process of performing a Gua sha massage on a portion to be treated of a subject using the Gua sha massage apparatus 1, to perform the Gua sha massage more safely while protecting the skin of the portion to be treated.

Here, the portion to be treated of the subject refers to a body part such as the face, hand, arm, or leg where a Gua sha massage may be performed by applying pressure to generate friction.

Also, the operator refers to a person who holds the Gua sha massage apparatus 1 and performs the Gua sha massage, and the subject refers to a person who receives the Gua sha massage from the operator.

In this case, the operator and the subject may be the same person or different persons.

The Gua sha massage apparatus 1 with a diagnostic function according to an embodiment of the present disclosure includes a main body 100 configured to generate friction by applying pressure while bringing it into contact with the skin of a portion to be treated of the subject.

In this case, the main body 100 is generally configured to include a handle 110 and a skin contact 120.

Meanwhile, the handle 110 provided in the main body 100 may have a bar or rod shape graspable by the operator.

In addition, the skin contact 120 has a structure in which a bar or rod shape extends from or is connected to the handle 110, and at least one contact surface 121 is frictionally in contact with the portion to be treated.

Such a skin contact 120 may be formed at various parts of the main body 100 according to the shape of the main body 100, and the main body 100 includes at least one such skin contact 120.

Meanwhile, for example, the main body 100 including the above-described handle 110 and skin contact 120 may have a donut shape with a through hole 101 through which the operator's finger passes (see FIG. 1). Alternatively, in another example, the main body 100 may have a knuckle shape in which a plurality of through holes 101 are formed to allow each finger of the operator to pass through.

The main body 100 constituting such a Gua sha massage apparatus 1 with a diagnostic function is not limited to any one shape as long as it has a shape graspable by the operator, and various shapes may be applied as long as it can generate friction by applying pressure to the skin of the subject.

In addition, such a main body 100 may be made of various materials such as synthetic resin, metal, or stone as needed.

Meanwhile, in an embodiment for explaining the present disclosure, the main body 100 of the Gua sha massage apparatus 1 with a diagnostic function is illustrated and described as having a quadrangular donut shape with a through hole 101 and four side surfaces including a top, bottom, left, and right (see FIG. 1).

In this case, the handle 110 is positioned at an upper portion of the main body 100 and has a graspable rod shape.

And the skin contact 120 forms at least one contact surface 121 that is in contact with the skin and is disposed at the left side, right side, and bottom of the above-described handle 110.

Accordingly, the skin contact 120 has respective contact surfaces 121, and includes a first skin contact 120a disposed at the bottom of the main body 100, a second skin contact 120b disposed at the left side of the handle 110 constituting the main body 100, and a third skin contact 120c disposed at the right side of the handle 110.

Meanwhile, the contact surfaces 121 of the first skin contact 120a, the second skin contact 120b, and the third skin contact 120c may be configured to form protruding portions of different shapes, and the operator may perform the Gua sha massage by selecting the skin contact 120 having contact surfaces 121 of various shapes according to a portion to be treated of the subject.

Meanwhile, the contact surface 121 of the skin contact 120 is preferably formed of a material that does not cause wounds to the skin of a portion to be treated while being in contact with the skin and generating friction due to applied pressure.

For example, the contact surface 121 may be formed at an end of the skin contact 120 using ceramic, silver, wood, red agate, jade, a plastic material, or the like.

The contact surface 121 of the skin contact 120 generates friction while moving on a portion to be treated according to the pressure applied through contact with the skin.

Meanwhile, the first skin contact 120a, the second skin contact 120b, and the third skin contact 120c constituting the skin contact 120 may further include, as needed, on one side thereof, an electrical muscle stimulation (EMS) stimulation module 140 configured to stimulate a muscle with a microcurrent or a medium-frequency current of a preset frequency, or a radio frequency (RF) stimulation module 150 configured to provide deep thermal stimulation using a frequency of about 1 MHz.

The EMS stimulation module 140 and the RF stimulation module 150 may employ known structures and functions, and a detailed description thereof is omitted so as not to obscure the gist of the present disclosure.

However, the above-described EMS stimulation module 140 and the RF stimulation module 150 may treat portions to be treated that experience significant pain according to control signals generated by the controller 240 of the diagnosis module 200 described below, thereby alleviating and improving the level of pain at the portion to be treated.

Meanwhile, the Gua sha massage apparatus 1 with a diagnostic function according to an embodiment of the present disclosure includes, on one side of the main body 100, a diagnosis module 200 configured to confirm the pain level at the portion to be treated, to perform the Gua sha massage treatment more safely while protecting the subject's skin.

The operator can confirm and record the pain level for each portion to be treated of the subject using the diagnosis module 200 described above, and perform Gua sha massage while applying appropriate pressure to each portion to be treated accordingly.

As a specific embodiment for this, the above-described diagnosis module 200 generally includes a pressure measurer 210, a database 220, a comparator 230, a controller 240, and a feedback generator 250.

First, the pressure measurer 210 serves to measure the pressure generated when the contact surface 121 of the skin contact 120 comes into contact with the skin of the portion to be treated, on one side of an internal space of the main body 100.

For example, the pressure measurer 210 is illustrated (see FIG. 3) as being provided on a protruding portion forming one side of the third skin contact 120c disposed on the right side of the handle 110; however, it is not limited thereto.

In other words, if the operator may generate pressure by bringing the pressure measurer 210 into contact with the skin of the portion to be treated, the pressure measurer 210 may, as needed, be formed on one side of the first skin contact 120a or the second skin contact 120b (see FIG. 1).

Referring again to FIG. 3, in an embodiment, the pressure measurer 210 forms a protruding side of the skin contact 120.

The pressure measurer 210 includes: a diagnostic member 211 integrated with the skin contact 120 and having a diagnostic surface 214 that contacts the portion to be treated; a load cell 212 disposed in an internal space of the skin contact 120, the load cell serving as a pressure measuring sensor to measure a pressure value transmitted through the diagnostic member 211; and an elastic member 213 disposed in the internal space of the skin contact 120 to support the load cell 212 and to accommodate displacement according to the pressure applied to the diagnostic member 211.

First, the diagnostic member 211 of the pressure measurer 210 generates pressure by being brought into contact with and pressed against the skin of the actual portion to be treated, and transfers the generated pressure to the load cell 212 serving as the pressure measuring sensor.

For this, the diagnostic member 211 is formed of an elastic material such as silicone or rubber and is integrally formed at an end of the skin contact 120. In addition, the diagnostic surface 214 of the diagnostic member 211 preferably has a rounded curved shape so as not to cause wounds to the skin when brought into contact with the portion to be treated.

Meanwhile, if necessary, the diagnostic member 211 may be made of a hard rigid material.

And the diagnostic member 211 may have a detachable coupling structure at an end of the skin contact 120 as needed for replacement of the pressure measuring sensor, or may be provided in an integral form.

Meanwhile, the diagnostic member 211 may be formed of two members having different hardnesses to facilitate the generation and transmission of pressure.

In other words, the diagnostic member 211 includes a first diagnostic member 211a having the diagnostic surface 214, and a second diagnostic member 211b disposed inside the first diagnostic member 211a to support the above-described pressure sensor and the elastic member 213.

In this case, the first diagnostic member 211a and the second diagnostic member 211b may be configured to have different hardnesses.

Meanwhile, the Gua sha massage apparatus 1 with a diagnostic function according to an embodiment of the present disclosure uses a load cell 212 as a pressure measuring sensor constituting the pressure measurer 210.

Such a load cell 212 measures pressure by using a resistance change rate of a strain gauge according to a load applied through the diagnostic member 211, and a known load cell 212 may be applied.

The load cell 212 is disposed with one side receiving a load generated by the diagnostic member 211 to measure a pressure value, and the other side supported while contacting a rigid wall or support member forming the internal space of the skin contact 120.

Since various known structures may be applied to the load cell 212, detailed descriptions of the function and configuration thereof will be omitted so as not to obscure the gist of the present disclosure.

However, since the load cell 212 generally precisely detects a low load, the Gua sha massage apparatus 1 with a diagnostic function according to an embodiment of the present disclosure may more accurately measure pressure according to the magnitude of force applied to the portion to be treated by applying the load cell 212 as the pressure measuring sensor.

And the elastic member 213 supporting the load cell 212 described above is disposed in the internal space of the skin contact 120 and accommodates displacement according to the pressure applied to the diagnostic member 211.

The elastic member 213 may be made of an elastic material such as silicone or rubber, and prevents the load cell 212 from being displaced from its position disposed inside the internal space of the skin contact 120.

The elastic member 213 may have a block shape filling the internal space of the skin contact 120, or a rib shape in which a plurality of bars are connected to each other or arranged independently.

As described above, the Gua sha massage apparatus 1 with the diagnostic function according to an embodiment of the present disclosure includes the pressure measurer 210 in the diagnosis module 200 to confirm the pain level of the subject using the pressure value generated at the portion to be treated.

In this case, the pressure measurer 210 is preferably disposed along a line in a direction the same as a pressure direction applied to a portion to be treated of a subject, so that the pressure value may be measured more accurately.

In addition, the above-described pressure measurer 210 may include at least one load cell 212.

Among a plurality of pressure values measured by a plurality of load cells 212, a value of the highest pressure or an average value of the pressure may be used as determination data as needed.

Subsequently, in the diagnosis module 200 of the Gua sha massage apparatus 1 with the diagnostic function according to the embodiment of the present disclosure, pressure value information measured by the above-described pressure measurer 210 is stored in the database 220.

In this case, the pressure value information stored in the database 220 is the pressure value measured by the pressure measurer 210.

In addition, a pressure value confirmed as a pressure at which the subject feels pain during the Gua sha massage treatment using the pressure value measured by the pressure measurer 210 may be stored as pain pressure value information.

Such a pain pressure value is confirmed through the comparator 230 described below and stored in the database 220 under the control of the controller 240.

Meanwhile, the comparator 230 determines the pain pressure value as described above and, during the Gua sha massage treatment, compares the above-described pain pressure value with the pressure value measured by the pressure measurer 210 to determine the pain level.

Through the numerical determination of the pressure value by the comparator 230 as described above, the operator may perform Gua sha massage treatment safely while protecting the skin of the portion to be treated by applying only an appropriate pressure that does not exceed the pain pressure value to the subject's portion to be treated.

Meanwhile, among the pressure values measured by the pressure measurer 210, one pressure value may be determined as a pain pressure value corresponding to pain felt at each portion to be treated of the subject by manual or automatic selection and stored.

For example, such a pain pressure value may be confirmed and stored as an average pressure value of pressure values measured for each unit of time while pressing the portion to be treated for a preset period of time, or a maximum pressure value among the pressure values measured while pressing the portion to be treated for the predetermined period of time, using the diagnosis module 200.

In addition, in another embodiment, when the operator arbitrarily presses the operation button 130 provided on one side of the main body 100 of the Gua sha massage apparatus 1 with the diagnostic function, the pressure value at the selected moment may be confirmed and stored as the pain pressure value.

The above-described operation button 130 is pressed when the subject receiving the Gua sha massage recognizes pain in the portion to be treated and gives a signal to the operator.

Preferably, the above-described pain pressure value may be an average pressure value of pressure values measured for each unit of time during confirmation of pain in the portion to be treated while pressing the portion to be treated for a preset period of time.

A Gua sha massage device is generally a device configured to apply friction to the skin of a portion to be treated to cause pain, and it is particularly important to clearly confirm the pain level of a subject undergoing treatment, that is, the degree to which the subject feels pain but can bear it without severe discomfort.

The reason why the pain pressure value is defined as the average pressure value of pressure values measured during pain confirmation of the portion to be treated for each unit of time while pressurizing the portion for the preset period of time is that the subject experiences a tolerable pain level which, however, may cause skin damage. Therefore, setting the average pressure value of the measured values as the pain pressure value may satisfy the subject's treatment requirements for Gua sha massage while simultaneously preventing skin damage.

Meanwhile, the controller 240 constituting the diagnosis module 200 applied to the Gua sha massage apparatus 1 with the diagnostic function according to an embodiment of the present disclosure generates a control signal according to the determination of the comparator 230 described above.

Such a control signal may be a control signal of the electrical muscle stimulation (EMS) stimulation module 140 described above, a control signal of the radio frequency (RF) stimulation module 150, or a feedback notification signal of the feedback generator 250 described below.

The controller 240 consists of a Micro Controller Unit (MCU).

The controller 240 may also control operations of other components (e.g., vibration motor 251, speaker 252, light-emitting lamp 253, numerical display 254, etc.) constituting the Gua sha massage apparatus 1 with the diagnostic function, as well as additionally configured components (see FIG. 4).

For example, the controller 240 may compare the stored pain pressure value with the pressure value measured by the pressure measurer 210, and, if the measured pressure value gradually exceeds the pain pressure value, control the vibration motor 251 to increase its vibration strength progressively.

In addition, the controller 240 may compare the stored pain pressure value with the pressure value measured by the pressure measurer 210, and, if the measured pressure value gradually exceeds the pain pressure value, control the speaker 252 to increase its sound progressively.

Further, the controller 240 may compare the stored pain pressure value with the pressure value measured by the pressure measurer 210, and, if the measured pressure value gradually exceeds the pain pressure value, control the light of the light-emitting lamp 253 to gradually increase in intensity or adjust the blinking frequency of the light to gradually become faster or slower.

Further, the controller 240 may compare the stored pain pressure value with the pressure value measured by the pressure measurer 210, and, if the measured pressure value gradually exceeds the pain pressure value, control the numerical display 254 to change the numerical indication accordingly.

Meanwhile, the Gua sha massage apparatus 1 with the diagnostic function according to an embodiment of the present disclosure may be configured to link the pressure value information stored in the database 220 of the diagnosis module 200 with an external smartphone or server (not shown) via a network.

As described above, the pressure values stored in the database 220 are digitized and based on the accumulated pressure value data for each subject or each portion to be treated, the operator can perform a safer Gua sha massage treatment by applying the most suitable pressure to the subject receiving the Gua sha massage.

Meanwhile, the above-described network may communicate with a smartphone or an external server via data communication such as 2G, 3G, 4G, WiFi, or LAN, or via Bluetooth communication.

Meanwhile, as described above, the Gua sha massage apparatus 1 with the diagnostic function according to an embodiment of the present disclosure compares, in the comparator 230, the pain pressure value stored in the database 220 with the pressure value measured by the pressure measurer 210 during the Gua sha massage treatment.

If the pressure value measured by the pressure measurer 210 exceeds the pain pressure value, the controller 240 generates feedback through the feedback generator 250 so that the operator or the subject can recognize it, and provides an external notification through a notification signal.

Accordingly, when performing a Gua sha massage using the Gua sha massage apparatus 1 with the diagnostic function, the operator may apply only an appropriate pressure to the portion to be treated of the subject, thereby protecting the subject's skin and enabling a safer massage treatment.

In other words, it is determined whether the pressure value measured by the pressure measurer 210 falls within a pain pressure value range, and when it is confirmed that a pressure exceeding the pain pressure value is applied, the feedback generator 250 generates feedback as a notification signal recognizable by the operator or the subject under the control of the controller 240.

If the pressure value measured by the pressure measurer 210 is determined by the comparator 142 to be equal to or less than an appropriate pressure value, it is recognized as a pressure value within a safe range and no feedback signal is generated.

Referring again to FIG. 4, the notification signal generated by the feedback generator 250 may be a vibration signal as shown in FIG. 4(a), a sound signal as shown in FIG. 4(b), a light emission signal using light emission as shown in FIG. 4(c), or a numerical display signal using a numerical display as shown in FIG. 4(d).

To this end, the Gua sha massage apparatus 1 with the diagnostic function may be provided with a vibration motor 251 on one side inside the main body 100 to generate the vibration signal as shown in FIG. 4(a).

In addition, the Gua sha massage apparatus 1 with the diagnostic function may be provided with a speaker 252 on one side of the main body 100 to generate the sound signal as shown in FIG. 4(b).

Further, the Gua sha massage apparatus 1 with the diagnostic function may be provided with a light-emitting lamp 253 on one side of the main body 100 to generate the light emission signal as shown in FIG. 4(c).

Meanwhile, the Gua sha massage apparatus 1 with the diagnostic function may be provided with a numerical display 254 on one side of the main body 100 to generate the numerical display signal as shown in FIG. 4(d).

In other words, the Gua sha massage apparatus 1 with the diagnostic function according to an embodiment of the present disclosure includes at least one of the vibration motor 251, the speaker 252, the light-emitting lamp 253, and the numerical display 254 on one side of the main body 100.

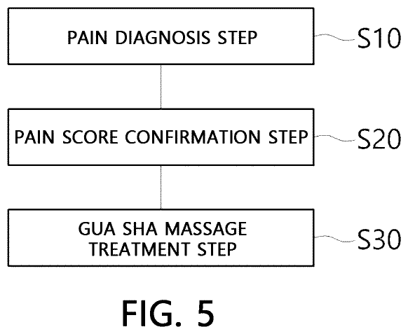

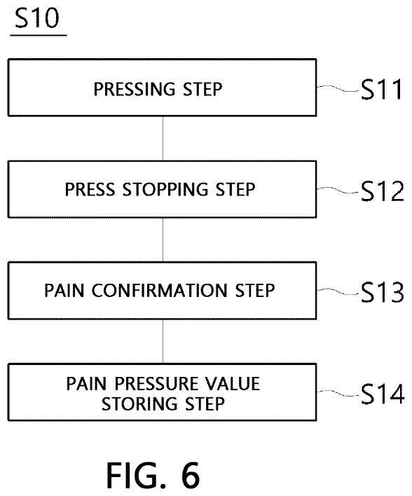

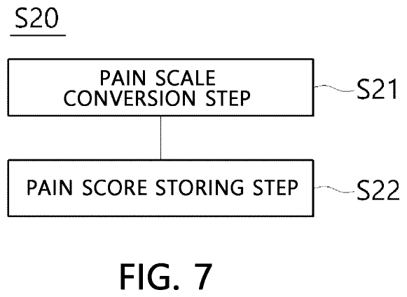

Meanwhile, FIG. 5 is a block diagram showing a Gua sha massage method using the Gua sha massage apparatus with the diagnostic function according to an embodiment of the present disclosure, FIG. 6 is a block diagram showing a pain diagnosis step in the Gua sha massage method using the Gua sha massage apparatus with the diagnostic function according to an embodiment of the present disclosure, and FIG. 7 is a block diagram showing a pain score confirmation step in the Gua sha massage method using the Gua sha massage apparatus with the diagnostic function according to an embodiment of the present disclosure.

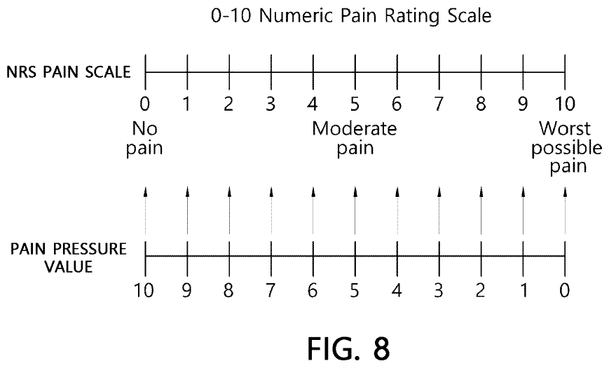

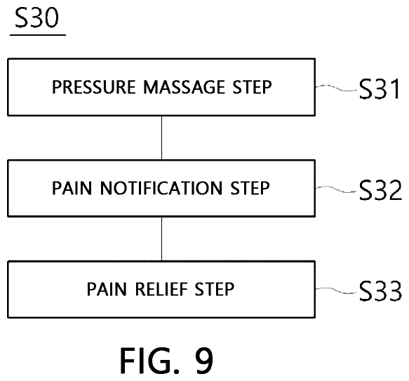

Further, FIG. 8 is a view showing an example of a pain scale chart applied to the pain score confirmation step in the Gua sha massage method using the Gua sha massage apparatus with the diagnostic function according to an embodiment of the present disclosure, and FIG. 9 is a block diagram showing a Gua sha massage treatment step in the Gua sha massage method using the Gua sha massage apparatus with the diagnostic function.

Referring to FIGS. 5 to 9, the Gua sha massage method using the above-described Gua sha massage apparatus 1 with the diagnostic function will be described in more detail as follows.

The Gua sha massage method using the Gua sha massage apparatus 1 with the diagnostic function according to an embodiment of the present disclosure enables a safer Gua sha massage to be performed while protecting the skin of the portion to be treated of the subject.

The subject receiving the Gua sha massage may experience different levels of pressure-induced pain depending on the pressure applied to each portion to be treated.

Accordingly, the Gua sha massage method according to an embodiment of the present disclosure first diagnoses and measures the level of pressure-induced pain at the subject's portion to be treated by using the diagnosis module 200 of the Gua sha massage apparatus 1 with the diagnostic function, and based on this, enables the operator to perform the Gua sha massage on the subject within a range where the subject does not experience severe pain.

As described above, the operator and the subject may be the same person or may be different persons.

Thus, the pressure values measured through the diagnosis module 200 are stored as pain pressure values for each portion to be treated and may be used as reference information when the subject receives the next Gua sha massage.

In addition, the subject may experience different pain levels at each portion to be treated depending on the body condition or health status on the day of receiving the Gua sha massage treatment. Accordingly, before receiving the Gua sha massage treatment, the subject can measure and confirm the pain pressure value through the diagnosis module 200, thereby allowing the Gua sha massage to be performed more safely.

The Gua sha massage method using the Gua sha massage apparatus with a diagnostic function according to an embodiment of the present disclosure generally includes a pain diagnosis step (step S10), a pain score confirmation step (step S20), and a Gua sha massage treatment step (step S30).

First, the pain diagnosis step (step S10) includes bringing the pressure measurer 210, which constitutes the diagnosis module 200 of the Gua sha massage apparatus 1 with the diagnostic function, into contact with the subject's portion to be treated and pressing the portion to be treated, and when pain occurs at the portion to be treated, storing the pressure value measured by the above-described pressure measurer 210 as the pain pressure value.

Through the pain diagnosis step (step S10), it is possible to confirm pressure value information that causes pain for each subject or portion to be treated, and this pressure value information may be stored as data and utilized as data for each subject or portion to be treated.

Specifically, referring to FIG. 6, the pain diagnosis step (step S10) includes: a pressing step (step S11) of bringing the pressure measurer 210 of the diagnosis module into contact with the subject's portion to be treated and pressing the portion to be treated using the pressure measurer 210; a press stopping step (step S12) of stopping the pressing when pain is confirmed at the portion to be treated; a pain confirmation step (step S13) of confirming the pain level of the subject for the pressure value measured in the press stopping step (step S12); and a pain pressure value storing step (step S14) of storing the pressure value confirmed in the pain confirmation step (step S13) as the pain pressure value.

In an embodiment, the pain pressure value storing step (step S14) may include storing, as the pain pressure value, an average pressure value of pressure values measured for each unit of time while pressing the portion to be treated for a preset period of time, or storing, as the pain pressure value, a maximum pressure value among the pressure values measured while pressing the portion to be treated for a preset period of time.

In another embodiment, the pain pressure value storing step (step S14) may include storing, as the pain pressure value, a selected pressure value when an operation button provided on one side of the main body 100 of the Gua sha massage apparatus 1 with the diagnostic function is pressed.

Subsequently, the pain score confirmation step (step S20) includes converting the pain pressure value confirmed in the above-described pain diagnosis step (S10) into a pain scale, scoring it, and storing the score.

Through the pain score confirmation step (step S20), the operator or the subject can confirm the level of pain corresponding to the pain pressure value on the pain scale, and the pain pressure value can also be used as a scale for assessing the health status of the subject.

Specifically, referring to FIG. 7, the pain score confirmation step (step S20) includes a pain scale conversion step (step S21) of confirming a pain score by comparing the above-described pain pressure value with a score in a pain scale chart; and a pain score storing step (step S22) of storing the confirmed pain score.

In this case, a commonly known pain scale chart capable of identifying various pain levels may be used as the pain scale chart.

For example, the numerical rating scale (NRS) pain scale as shown in FIG. 8 may be applied as the pain scale.

For example, if the pressure value measured by the load cell 212 of the pressure measurer 210 is indicated on a scale of 0 to 10, a pressure value of 10 may indicate that the corresponding portion to be treated does not feel pain even when strong pressure is applied, whereas a pressure value of 0 may indicate that the corresponding portion to be treated feels strong pain even under slight pressure.

The level of pain at the portion to be treated can be objectively identified by comparing the pain pressure values at each portion to be treated that cause pain with the values of the pain scale chart and confirming the pain value.

Such pain scores are digitized through the pain score storing step (step S22) and stored in the comparator 230 of the diagnosis module 200.

Meanwhile, the pain scores may be further compared with the pressure values measured during the Gua sha massage treatment as needed. The level of pain corresponding to changes in the pressure values during the Gua sha massage treatment may be confirmed by the operator or the subject as a numerical display signal through the numerical display 254.

Meanwhile, the above-described NRS pain scale chart is merely an example and is not limiting. If the level of pain can be represented by an objective numerical value, a Visual Analog Scale (VAS) or various other pain scale charts may also be applied.

Next, the Gua sha massage treatment step (step S30) is a step of performing the Gua sha massage within a range of pressure values equal to or less than the pain pressure value described above, while allowing the pressure measurer 210 to measure the pressure.

Specifically, the Gua sha massage treatment step (step S30) includes a pressure massage step (step S31) of pressing while bringing the skin contact 120 into contact with the portion to be treated; and a pain notification step (step S32) of generating a notification signal recognizable by the operator or a subject through the feedback generator 250 when the pressure value measured by the pressure measurer 210 is equal to or greater than the stored pain pressure value.

The notification signal generated by the feedback generator 250 may be generated as any one of a numerical display signal, a sound signal, a vibration signal, or a light-emitting signal.

For example, referring to FIG. 4(a), when the pressure value measured by the pressure measurer 210 is greater than the pain pressure value, a vibration signal may be generated by producing vibration using a vibration motor 251 as the notification signal.

In this case, while comparing the stored pain pressure value with the pressure value measured by the pressure measurer 210, if the measured pressure value gradually exceeds the pain pressure value, the vibration motor 251 may be controlled to gradually increase its vibration intensity.

Another example, referring to FIG. 4(b), when the pressure value measured by the pressure measurer 210 is greater than the pain pressure value, a sound signal may be generated as the notification signal by producing a preset sound using a speaker 252.

In this case, while comparing the stored pain pressure value with the pressure value measured by the pressure measurer 210, if the measured pressure value gradually exceeds the pain pressure value, the sound from the speaker 252 may be controlled to gradually increase in volume.

Another example, referring to FIG. 4(c), when the pressure value measured by the pressure measurer 210 is greater than the pain pressure value, a light-emitting signal may be generated as the notification signal by blinking a light-emitting lamp 253.

In this case, while comparing the stored pain pressure value with the pressure value measured by the pressure measurer 210, if the measured pressure value gradually exceeds the pain pressure value, the light from the light-emitting lamp 253 may be controlled to gradually increase in intensity, or the blinking frequency of the light may be controlled to gradually increase or decrease.

Another example, referring to FIG. 4(d), when the pressure value measured by the pressure measurer 210 is greater than the pain pressure value, a numerical display signal may be generated as the notification signal by displaying the converted pain value on a numerical display 254.

In this case, while comparing the stored pain pressure value with the pressure value measured by the pressure measurer 210, if the measured pressure value gradually exceeds the pain pressure value, the numerical display shown on the numerical display 254 may be controlled to change.

Meanwhile, according to the Gua sha massage method of the present embodiment, the subject may recognize muscle tension, skin resistance, and the like by themselves through the notification signals generated by the above-described feedback generator 250.

Since the operator or subject perceives the level and area of pain through visual, auditory, and tactile senses during the Gua sha massage treatment, the concentration on the Gua sha massage treatment is naturally increased, thereby facilitating pain management of the portion to be treated through the Gua sha massage.

Meanwhile, the Gua sha massage treatment step (step S30) may further include a pain relief step (step S33) of operating any one of an electrical muscle stimulation (EMS) stimulation module 140 configured to stimulate a muscle with a microcurrent or a medium-frequency current of a preset frequency, or a radio frequency (RF) stimulation module 150 configured to provide deep thermal stimulation using a frequency, to stimulate the portion to be treated when a notification signal is generated and the subject feels strong pain at the portion to be treated.

As described above, by performing the pain relief step (step S33) using the EMS stimulation module 140 or the RF stimulation module 150, a treatment function can be performed on the portion to be treated where strong pain is felt, thereby alleviating the level of pain. This can further maximize the effect of the Gua sha massage treatment.

As described above, the Gua sha massage apparatus 1 with the diagnostic function according to an embodiment of the present disclosure and the Gua sha massage method using the same may perform a Gua sha massage within a range of pain pressure values measured by the pressure measurer 210 of the diagnosis module 200, thereby protecting the subject's portion to be treated and enabling safe massage treatment.

In addition, when a pressure value equal to or greater than the pain pressure value occurs, feedback recognizable by the operator or the subject may be generated and provided through the feedback generator 250 of the diagnosis module 200, thereby significantly reducing the degree and likelihood of bruising that may occur at the portion to be treated during the Gua sha massage, protecting the skin at the portion to be treated, and improving safety.

Furthermore, the feedback generator 250 may prevent excessive use of the Gua sha massage apparatus by providing the operator or the subject with the notification signal of any one of sound, light, or vibration, and may also prevent damage to the subject's portion to be treated.

In addition, the pain pressure value may be set through manual or automatic operation, allowing the operator to perform the Gua sha massage treatment within a desired pressure range.

Meanwhile, the pressure measurer 210 of the diagnosis module 200 may accurately measure the pressure value by using the load cell 212 to measure pressure.

Also, the EMS stimulation module 140 and the RF stimulation module 150 may be selectively used under control, thereby treating a portion to be treated where pain is strongly felt to alleviate the degree of pain and improve the painful area.

Meanwhile, the pain pressure value suitable for each subject for each portion to be treated may be confirmed, and the pressure values stored in the database 220 may be digitized, thereby enabling a safer Gua sha massage treatment at the pressure most suitable for the subject based on the accumulated pressure value data.

Moreover, since the operator or subject perceives the level and area of pain through visual, auditory, and tactile senses during the Gua sha massage treatment, the concentration on the Gua sha massage treatment is naturally increased, thereby facilitating management of the painful area through the Gua sha massage.

Meanwhile, the stored pain pressure values may be utilized as individual data for each subject, providing useful feedback for the subject during the next Gua sha massage treatment, thereby enabling effective Gua sha massage treatment and increasing satisfaction with the use of the Gua sha massage apparatus.

Although exemplary embodiments of the present disclosure have been described, the spirit of the present disclosure is not limited to the embodiments set forth herein. Those of ordinary skill in the art who understand the spirit of the present disclosure may easily propose other embodiments through supplement, change, removal, addition, etc. of elements within the same scope of spirit, but the embodiments will be also within the scope of the present disclosure.

1. A Gua sha massage apparatus with a diagnostic function, comprising: a main body including a graspable handle and at least one skin contact having a contact surface configured to be frictionally in contact with a portion to be treated,
the main body comprising:
a diagnosis module configured to measure a pressure value generated upon contact with the portion to be treated and to confirm a level of pain at the portion to be treated by using the pressure value,
the diagnosis module comprising:
at least one pressure measurer configured to measure the pressure value generated upon contact with the portion to be treated;
a database configured to store information on a pain pressure value confirmed by using the pressure value measured by the pressure measurer and the pressure value measured by the pressure measurer;
a comparator configured to determine the pain pressure value and compare the pain pressure value with the pressure value measured by the pressure measurer to determine a numerical value;
a controller configured to generate a control signal based on the determination of the comparator; and
a feedback generator configured to generate a notification signal recognizable by an operator based on the control signal from the controller.

2. The Gua sha massage apparatus of claim 1, wherein the pressure measurer includes:
a diagnostic member formed on one side of the skin contact, the diagnostic member having a diagnostic surface configured to come into contact with the portion to be treated and being integrally provided with the skin contact;
a pressure sensor disposed in an internal space of the skin contact and configured to measure the pressure value transmitted through the diagnostic member; and
an elastic member disposed in the internal space of the skin contact and configured to accommodate a displacement according to a pressure applied to the diagnostic member and to support the pressure sensor.

3. The Gua sha massage apparatus of claim 2, wherein the pressure sensor includes a load cell.

4. The Gua sha massage apparatus of claim 2, wherein the diagnostic member includes:
a first diagnostic member having the diagnostic surface; and
a second diagnostic member disposed inside the first diagnostic member and configured to support the pressure sensor and the elastic member,
wherein the first diagnostic member and the second diagnostic member have different hardnesses.

5. The Gua sha massage apparatus of claim 1, wherein the pressure measurer is disposed along a line in a direction the same as a pressure direction applied to a portion to be treated of a subject.

6. The Gua sha massage apparatus of claim 1, wherein the main body has a donut shape or a knuckle shape with a through hole through which a finger of a subject passes.

7. The Gua sha massage apparatus of claim 1, wherein the pain pressure value is
an average pressure value of pressure values measured for each unit of time while pressing the portion to be treated for a preset period of time, or
a maximum pressure value among pressure values measured while pressing the portion to be treated for a preset period of time.

8. The Gua sha massage apparatus of claim 1, wherein the main body further includes an operation button on one side thereof, and
the pain pressure value is a pressure value measured by the pressure measurer when the operation button is operated.

9. The Gua sha massage apparatus of claim 1, wherein the main body includes at least one of a vibration motor, a speaker, a light-emitting lamp, and a numerical display on one side thereof.

10. The Gua sha massage apparatus of claim 1, wherein the information stored in the database is linked to an external smartphone or server via a network.

11. The Gua sha massage apparatus of claim 1, wherein the skin contact includes:
an electrical muscle stimulation (EMS) stimulation module configured to stimulate a muscle with a microcurrent or a medium-frequency current of a preset frequency, or
a radio frequency (RF) stimulation module configured to provide deep thermal stimulation using a frequency.

12. A Gua sha massage method using a Gua sha massage apparatus with a diagnostic function, the method comprising:
a pain diagnosis step of bringing a pressure measurer into contact with a portion to be treated, pressing the portion to be treated, and storing a pressure value measured by the pressure measurer as a pain pressure value when pain occurs at the portion to be treated;
a pain score confirmation step of converting the pain pressure value into a pain scale, scoring it and storing the score; and
a Gua sha massage treatment step of performing a Gua sha massage within a range of pressure values equal to or less than the pain pressure value.

13. The Gua sha massage method of claim 12, wherein the pain diagnosis step includes:
a pressing step of bringing the pressure measurer into contact with the portion to be treated of a subject and pressing the portion to be treated using the pressure measurer;
a press stopping step of stopping the pressing when pain is confirmed at the portion to be treated;
a pain confirmation step of confirming the pain level of the subject for the pressure value measured in the press stopping step; and
a pain pressure value storing step of storing the pressure value confirmed in the pain confirmation step as the pain pressure value.

14. The Gua sha massage method of claim 13, wherein the pain pressure value storing step includes:
storing, as the pain pressure value, an average pressure value of pressure values measured for each unit of time while pressing the portion to be treated for a preset period of time, or
storing, as the pain pressure value, a maximum pressure value among pressure values measured while pressing the portion to be treated for a preset period of time.

15. The Gua sha massage method of claim 13, wherein the pain pressure value storing step includes:
storing, as the pain pressure value, a selected pressure value when an operation button provided on one side of the main body of the Gua sha massage apparatus with the diagnostic function is pressed.

16. The Gua sha massage method of claim 12, wherein the pain score confirmation step includes:
a pain scale conversion step of confirming a pain score by comparing the pain pressure value with a score in a pain scale chart; and
a pain score storing step of storing the pain score.

17. The Gua sha massage method of claim 16, wherein the pain scale chart employs a numerical rating scale (NRS) pain scale.

18. The Gua sha massage method of claim 12, wherein the Gua sha massage treatment step includes:
a pressure massage step of pressing while bringing the skin contact into contact with the portion to be treated; and
a pain notification step of generating a notification signal recognizable by the operator or a subject through the feedback generator when the pressure value measured by the pressure measurer is equal to or greater than the stored pain pressure value.

19. The Gua sha massage method of claim 18, wherein the Gua sha massage treatment step further includes:
a pain relief step of operating any one of an electrical muscle stimulation (EMS) stimulation module configured to stimulate a muscle with a microcurrent or a medium-frequency current of a preset frequency, or a radio frequency (RF) stimulation module configured to provide deep thermal stimulation using a frequency to stimulate the portion to be treated.

20. The Gua sha massage method of claim 18, wherein the notification signal is generated as any one of a numerical display signal, a sound signal, a vibration signal, or a light-emitting signal.

21. The Gua sha massage method of claim 20, wherein in the pain notification step, when the pressure value measured by the pressure measurer increases to be equal to or greater than the stored pain pressure value, the numerical display signal indicates a change in numerical value, the sound signal indicates a change in volume, the vibration signal indicates a change in intensity, and the light emission signal indicates a change in blinking frequency of light.
